(19) United States
(12) Patent Application Publication
(10) Pub. No.: US 2008/0312444 A1
(43) Pub. Date: Dec. 18, 2008

(54) Process for Preparing Bicyclic Compounds

(76) Inventors: Sergio Bacchi, Verona (IT); Monica Delpogetto, Verona (IT); Simone Guelfi, Verona (IT); Alcide Perboni, Verona (IT); Arianna Ribecai, Verona (IT); Paolo Stabile, Verona (IT); Marsia Tampieri, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/910,985

(22) PCT Filed: Apr. 6, 2006
(86) PCT No.: PCT/EP2006/003531

§ 371 Date: Aug. 1, 2008

(30) Foreign Application Priority Data
Apr. 8, 2005 (GB) 0507198.0

(51) Int. Cl. C07D 471/04; C07D 207/22

(52) U.S. Cl. 546/113; 548/558

(57) ABSTRACT

The present invention relates to a novel process for preparing compounds of formula (IA), which are potent and specific antagonists of corticotropin-releasing factor (CRF) receptors, from intermediate compounds of formula (I), by a coupling reaction catalysed by copper.

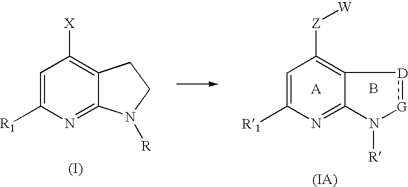

The present invention relates to a novel process and an intermediate compound, useful for preparing key intermediates in the synthesis of various bicyclic compounds, which are potent and specific antagonists of corticotropin-releasing factor (CRF) receptors.

The first corticotropin-releasing factor (CRF) was isolated from ovine hypothalami and identified as a 41-amino acid peptide (Vale et al., Science 213: 1394-1397, 1981).

CRF has been found to produce profound alterations in endocrine, nervous and immune system function. CRF is believed to be the major physiological regulator of the basal and stress-release of adrenocorticotropic hormone (“ACTH”), Bendorphin and other proopiomelanocortin (“POMC”)-derived peptides from the anterior pituitary (Vale et al., Science 213: 1394-1397, 1981).

In addition to its role in stimulating the production of ACTH and POMC, CRF appears to be one of the pivotal central nervous system neurotransmitters and plays a crucial role in integrating the body's overall response to stress.

Administration of CRF directly to the brain elicits behavioral, physiological and endocrine responses identical to those observed for an animal exposed to a stressful environment. Accordingly, clinical data suggests that CRF receptor antagonists may represent novel antidepressant and/or anxiolytic drugs that may be useful in the treatment of the neuropsychiatric disorders manifesting hypersecretion of CRF.

The present invention relates to a novel process for preparing compounds of formula (IA), as disclosed in WO 04/094420, starting from key intermediates of general formula (I),

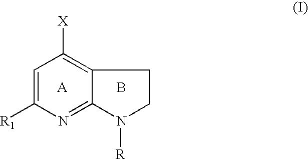

The variables R, R₁, and X may be defined as follows, but compounds of formula (I) are useful in the preparation of various bicyclic CRF antagonists which include, but are not limited to, those described in WO 95/10506, WO 04/094420, WO 03/008412 and WO 95/33750, in which the meaning of R, R₁, and X may be different.

In compounds of formula (I) R, R₁, and X may have the following meanings:
R is aryl or heteroaryl, each of which may be substituted by 1 to 4 groups J selected from:
   halogen, C1-C6 alkyl, C1-C6 alkoxy, halo C1-C6 alkyl, C2-C6 alkenyl, C2-C6 alkynyl, halo C1-C6 alkoxy, —C(O)R₂, nitro, hydroxy, —NR₃R₄, cyano, and or a group Z;
R₁ is hydrogen, C3-C7 cycloalkyl, C1-C6 alkyl, C1-C6 alkoxy, C1-C6 thioalkyl, C2-C6 alkenyl, C2-C6 alkynyl, halo C1-C6 alkyl, halo C1-C6 alkoxy, halogen, NR₃R₄ or cyano;
R₂ is a C1-C4 alkyl, —OR₃ or —NR₃R₄;
R₃ is hydrogen or C1-C6 alkyl;
R₄ is hydrogen or C1-C6 alkyl;
R₅ is a C1-C6 alkyl, halo C1-C6 alkyl, C1-C6 alkoxy, halo C1-C6 alkoxy, C3-C7 cycloalkyl, hydroxy, halogen, nitro, cyano, —NR₃R₄; —C(O)R₂;
X is an halogen.

Compounds of formula (IA), as disclosed WO 04/094420, have the following structure

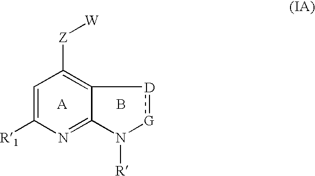

wherein
the dashed line may represent a double bond;
R′ corresponds to R;
R′₁ corresponds to R1;
R′₂ corresponds to R2;
R′₃ corresponds to R3;
R′₄ corresponds to R4;
R′₅ corresponds to R5;
R′₆ is a C1-C6 alkyl, halo C1-C6 alkyl, C1-C6 alkoxy, halo C1-C6 alkoxy, C3-C7 cycloalkyl, hydroxy, halogen, nitro, cyano, —NR′₃R′₄; —C(O)R′₂;
R′₇ is hydrogen, C1-C6 alkyl, halogen or halo C1-C6 alkyl;
R′₈ is hydrogen, C3-C7 cycloalkyl, C1-C6 alkyl, C2-C6 alkenyl, C2-C6 alkynyl, NR′₃R′₄ or cyano;
R′₉ is hydrogen, C3-C7 cycloalkyl, C1-C6 alkyl, C2-C6 alkenyl, C2-C6 alkynyl, NR′₃R′₄ or cyano;
R′₁₀ is hydrogen, C3-C7 cycloalkyl, C1-C6 alkyl, C2-C6 alkenyl, C2-C6 alkynyl, NR′₃R′₄ or cyano;
R′₁₁ is hydrogen, C3-C7 cycloalkyl, C1-C6 alkyl, C2-C6 alkenyl, C2-C6 alkynyl, NR′₃R′₄ or cyano;
R′₁₂ is R′₃ or —C(O)R′₂;
D is CR′₈R′₉ or is CR′₈ when double bonded with G;
G is CR′₁₀R′₁₁ or is CR′₁₀ when double bonded with D or is CR′₁₀ when double bonded with X when X is carbon;
W is a 4-8 carbocyclic membered ring, which may be saturated or may contain one to three double bonds, and
   in which:
      one carbon atom is replaced by a carbonyl or S(O)ₘ; and
      one to four carbon atoms may optionally be replaced by oxygen, nitrogen or NR′₁₂, S(O)ₘ, carbonyl, and such ring may be further substituted by 1 to 8 R′₆ groups;
Z is a 5-6 membered heterocycle, which may be substituted by 1 to 8 R′₅ groups;

m is an integer from 0 to 2.

Representative ring of the W definition include, but are not limited to, the following structure and derivatives:

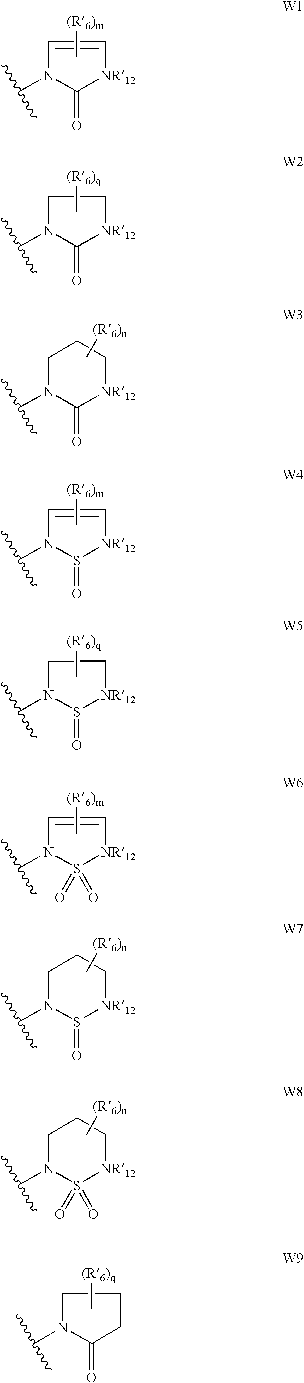

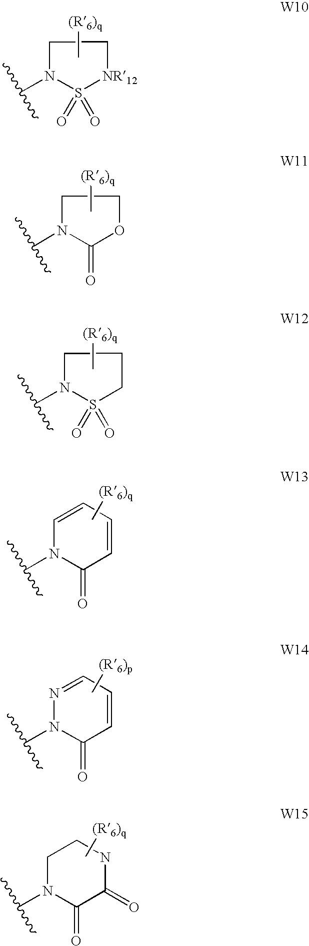

in which:
W1 represents a 1,3-dihydro-2H-imidazol-2-one derivative;
W2 represents a imidazolidin-2-one derivative;
W3 represents a tetrahydropyrimidin-2(1H)-one derivative;
W4 represents a 2,5-dihydro-1,2,5-thiadiazole 1-oxide derivative;
W5 represents a 1,2,5-thiadiazolidine 1-oxide derivative;
W6 represents a 2,5-dihydro-1,2,5-thiadiazole 1,1-dioxide derivative;
W7 represents a 1,2,6-thiadiazinane 1-oxide derivative;
W8 represents a 1,2,6-thiadiazinane 1,1-dioxide derivative;
W9 represents a pyrrolidin-2-one derivative;
W10 represents a 2,5-dihydro-1,2,5-thiadiazolidine 1,1-dioxide derivative;
W11 represents a 1,3-oxazolidin-2-one derivative;
W12 represents a isothiazolidine 1,1-dioxide derivative;
W13 represents a 2(1H)-pyridinone derivative;
W14 represents a 3(2H)-pyridazinone;
W15 represents a 2,3-piperazinedione derivative; and q is an integer from 0 to 4, n is an integer from 0 to 6, p is an integer from 0 to 3 and m, R′₆ and R′₁₂ are defined as above.

In another aspect the present invention provides a process useful for the preparation of compounds of formula (IIA):

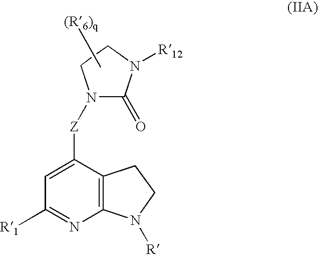

They correspond to compounds of formula (IA), in which W corresponds to a W2 derivative D and G are —CH₂ and R′, R′₁, R′₆, R′₁₂, and Z have the meanings as previously defined.

In a further aspect the present invention provides a process useful for the preparation of compounds of formula (IIIA), corresponding to compounds of formula (IIA) in which R′1 is —CH3, R′ is a phenyl derivative, Z is a pyrazolyl derivative.

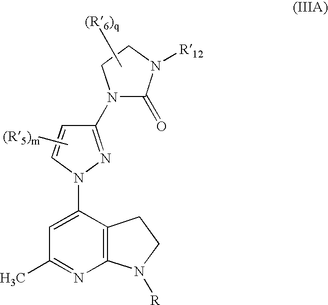

The term C1-C6 alkyl as used herein as a group or a part of the group refers to a linear or branched alkyl group containing from 1 to 6 carbon atoms; examples of such groups include methyl, ethyl, propyl, isopropyl, n-butyl, isobutyl, tert butyl, pentyl or hexyl.

The term C3-C7 cycloalkyl group means a non aromatic monocyclic hydrocarbon ring of 3 to 7 carbon atom; examples of such groups include cyclopropyl, cyclobutyl, cyclopentyl, cyclohexyl or cycloheptyl; while unsaturated cycloalkyls include cyclopentenyl and cyclohexenyl, and the like.

The term halogen refers to a fluorine, chlorine, bromine or iodine atom.

The term halo C1-C6 alkyl, or halo C1-C2 alkyl means an alkyl group having one or more carbon atoms and wherein at least one hydrogen atom is replaced with halogen such as for example a trifluoromethyl group and the like.

The term C1-C6 thioalkyl may be a linear or a branched chain thioalkyl group, for example thiomethyl, thioethyl, thiopropyl, thioisopropyl, thiobutyl, thiosec-butyl, thiotert-butyl and the like.

The term C2-C6 alkenyl defines straight or branched chain hydrocarbon radicals containing one or more double bond and having from 2 to 6 carbon atoms; examples of such groups include ethenyl, 2-propenyl, 3-butenyl, 2-butenyl, 2-pentenyl, 3-pentenyl, 3-methyl-2-butenyl or 3-hexenyl and the like.

The term C1-C6 alkoxy group may be a linear or a branched chain alkoxy group; examples of such groups include methoxy, ethoxy, propoxy, prop-2-oxy, butoxy, but-2-oxy or methylprop-2-oxy and the like.

The term halo C1-C6 alkoxy group may be a C1-C6 alkoxy group as defined before substituted with at least one halogen; examples of such groups include OCHF₂ or OCF₃.

The term C2-C6 alkynyl defines straight or branched chain hydrocarbon radicals containing one or more triple bond and having from 2 to 6 carbon atoms including acetylenyl, propynyl, 1-butynyl, 1-pentynyl, 3-methyl-1-butynyl and the like.

The term aryl means an aromatic carbocyclic moiety such as phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono- and bicyclic ring systems.

Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, and benzodioxolyl.

The term 5-6 membered heterocycle means, according to the above definition, a 5-6 monocyclic heterocyclic ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized. Heterocycles include heteroaryls as defined above. The heterocycle may be attached via any heteroatom or carbon atom. Thus, the term includes (but is not limited to) morpholinyl, pyridinyl, pyrazinyl, pyrazolyl, thiazolyl, triazolyl, imidazolyl, oxadiazolyl, oxazolyl, isoxazolyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, and the like.

In one aspect the present invention provides a process for preparing compounds of formula (IA) starting from compounds of formula (I), by a coupling reaction catalysed by copper

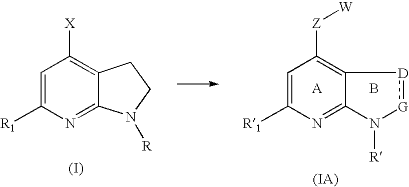

In one embodiment of the present invention the coupling reaction, similar to the Goldberg reaction, may be performed according to the following procedure.

A solution of a suitable copper catalyst selected in the group consisting from: CuI, CuBr, Cu₂Br, Cu(AcO)₂, Cu₂O; and a suitable ligand selected in the group consisting from: cis- or trans-N,N′-dimethyl-1,2-cyclohexanediamine, a mixture of cis- and trans-N,N′-dimethyl-1,2-cyclohexanediamine, cis- or trans-1,2-cyclohexanediamine, a mixture of cis- and trans-1,2-cyclohexanediamine, N,N′-dimethyl-1,2-diaminoethane, NN,N′N′-tetramethyl-1,2-diaminoethane, ethanolamine, 1,10-phenantroline, triphenylphosphine, BINAP, Acac; is prepared in a suitable solvent selected among polar aprotic solvents as defined above, or toluene, dioxane, 1,2-bis(methyloxy)ethane.

Then an inorganic or organic base as defined above is added followed by the reactive derivative of the upper residue (—W-Z) and the suitable intermediate compound (I). The resulting mixture is then kept at a temperature ranging from 80° to 150° C. for 4-48 hr. The mixture is then cooled at the end and worked as usual in order to provide a two layers mixture. The organic layer is constituted by a suitable organic solvent as described above. A suitable solvent may be added for improving the precipitation.

In one aspect the present invention provides a process for preparing the following compounds:
1-{1-[1-(4-Methoxy-2-methylphenyl)-6-methyl-2,3-dihydro-1H-pyrrolo[2,3-b]pyridin-4-yl]-1H-pyrazol-3-yl}imidazolidin-2-one;
1-{1-[1-(4-Methoxy-2-methylphenyl)-6-methyl-2,3-dihydro-1H-pyrrolo[2,3-b]pyridin-4-yl]-1H-pyrazol-3-yl}-3-methylimidazolidin-2-one;
1-{1-[1-(2,4-Dichlorophenyl)-6-methyl-2,3-dihydro-1H-pyrrolo[2,3-b]pyridin-4-yl]-1H-pyrazol-3-yl}imidazolidin-2-one;
1-Acetyl-3-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-Acetyl-3-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-(1-{1-[4-(Ethyloxy)-2-methylphenyl]-6-methyl-2,3-dihydro-1H-pyrrolo[2,3-b]pyridin-4-yl}-1H-pyrazol-3-yl)-2-imidazolidinone;
1-[1-(6-Methyl-1-{2-methyl-4-[(1-methylethyl)oxy]phenyl}-2,3-dihydro-1H-pyrrolo[2,3-b]pyridin-4-yl)-1H-pyrazol-3-yl]-2-imidazolidinone;
1-[1-(6-Methyl-1-{2-methyl-4-[(trifluoromethyl)oxy]phenyl}-2,3-dihydro-1H-pyrrolo[2,3-b]pyridin-4-yl)-1H-pyrazol-3-yl]-2-imidazolidinone;
1-(6-{6-Methyl-1-[2-methyl-4-(methyloxy)phenyl]-2,3-dihydro-1H-pyrrolo[2,3-b]pyridin-4-yl}-2-pyridinyl)-2-imidazolidinone;
1-(4-{6-Methyl-1-[2-methyl-4-(methyloxy)phenyl]-2,3-dihydro-1H-pyrrolo[2,3-b]pyridin-4-yl}-2-pyrimidinyl)-2-imidazolidinone;
1-(2-{6-Methyl-1-[2-methyl-4-(methyloxy)phenyl]-2,3-dihydro-1H-pyrrolo[2,3-b]pyridin-4-yl}-4-pyrimidinyl)-2-imidazolidinone;
1-(1-{6-Methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-(3-{6-Methyl-1-[2-methyl-4-(methyloxy)phenyl]-2,3-dihydro-1H-pyrrolo[2,3-b]pyridin-4-yl}phenyl)-2-imidazolidinone;
1-(5-Methyl-1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-{1-[1-(4-Methoxy-2-methylphenyl)-6-methyl-2,3-dihydro-1H-pyrrolo[2,3-b]pyridin-4-yl]-1H-pyrazol-3-yl}pyrrolidin-2-one;
1-{1-[1-(4-Methoxy-2-methylphenyl)-6-methyl-2,3-dihydro-1H-pyrrolo[2,3-b]pyridin-4-yl]-1H-pyrazol-3-yl}tetrahydropyrimidin-2(1H)-one;
3-(1-{6-Methyl-1-[2-methyl-4-(methyloxy)phenyl]-2,3-dihydro-1H-pyrrolo[2,3-b]pyridin-4-yl}-1H-pyrazol-3-yl)-1,3-oxazolidin-2-one;
Methyl 5-(1-{6-methyl-1-[2-methyl-4-(methyloxy)phenyl]-2,3-dihydro-1H-pyrrolo[2,3-b]-pyridin-4-yl}-1H-pyrazol-3-yl)-1,2,5-thiadiazolidine-2-carboxylate 1,1-dioxide);
4-[3-(1,1-Dioxido-1,2,5-thiadiazolidin-2-yl)-1H-pyrazol-1-yl]-6-methyl-1-[2-methyl-4-(methyloxy)phenyl]-2,3-dihydro-1H-pyrrolo[2,3-b]pyridine;
4-[3-(1,1-Dioxido-2-isothiazolidinyl)-1H-pyrazol-1-yl]-6-methyl-1-[2-methyl-4-(methyloxy)phenyl]-2,3-dihydro-1H-pyrrolo[2,3-b]pyridine;
3-Methyl-1-(1-{6-methyl-1-[2-methyl-4-(methyloxy)phenyl]-2,3-dihydro-1H-pyrrolo[2,3-b]pyridin-4-yl}-1H-pyrazol-3-yl)-2(1H)-pyridinone;
2-(1-{6-Methyl-1-[2-methyl-4-(methyloxy)phenyl]-2,3-dihydro-1H-pyrrolo[2,3-b]pyridin-4-yl}-1H-pyrazol-3-yl)-3(2H)-pyridazinone;
1-(1-{6-Methyl-1-[2-methyl-4-(methyloxy)phenyl]-2,3-dihydro-1H-pyrrolo[2,3-b]pyridin-4-yl}-1H-pyrazol-3-yl)-1,3-dihydro-2H-imidazol-2-one;
1-(1-{6-Methyl-1-[2-methyl-4-(methyloxy)phenyl]-1H-pyrrolo[2,3-b]pyridin-4-yl}-1H-pyrazol-3-yl)-2-imidazolidinone;
3-Methyl-4-[6-methyl-4-(3-thiazol-2-yl-pyrazol-1-yl)-2,3-dihydro-1H-pyrrolo[2,3-b]pyridin-1-yl]-benzonitrile;
1-(4-Methoxy-2-methyl-phenyl)-6-methyl-4-(3-thiazol-2-yl-pyrazol-1-yl)-2,3-dihydro-1H-pyrrolo[2,3-b]pyridine.

In one aspect, the present invention provides the preparation of 1-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone and 1-(1-{6-methyl-1-[2-methyl-4-(trifluoromethyloxy)phenyl]-2,3-dihydro-1H-pyrrolo[2,3-b]pyridin-4-yl}-1H-pyrazol-3-yl)-2-imidazolidinone which are reported in the Experimental section as illustrative of the procedure object of the present invention.

In one aspect, the present invention provides the new compounds of formula (VII). The compounds of formula (VII) are intermediates in the process for the preparation of compounds of formula (I), according to the following Scheme 1:

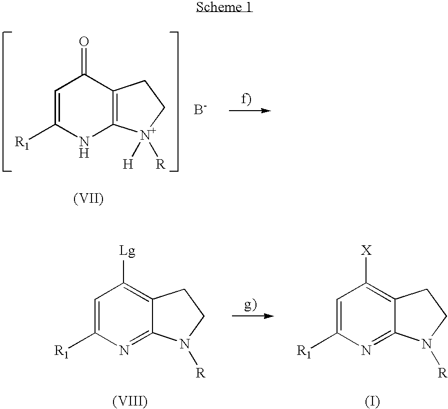

wherein R, R₁, and X are defined as above, and Lg is a leaving group selected among the reactive derivatives of an alkylsulphonic acid and
step f stands for the formation of a reactive derivative of the hydroxy pyridine of compounds (VII);
step g stands for nucleophilic displacement of the reactive derivative of compounds (VIII) to give the halogenated compounds (I).

Step f stands for the formation of a reactive derivative (i.e. a leaving group, Lg) of the hydroxy pyridine. The leaving group may be a reactive derivative of an alkylsulphonic acid, which includes but it is not limited to mesylate, tosylate, triflate.

To a suspension of intermediate compound (VII) in a suitable solvent which includes, but it is not limited to, chlorinated solvents (e.g. dichloromethane), an inorganic base in aqueous solution is added in order to provide the corresponding salt.

The suitable inorganic base may be selected from the group consisting of: sodium carbonate, sodium hydrogen carbonate, potassium carbonate, potassium hydrogen carbonate, sodium hydroxyde, potassium hydroxyde.

The salt so formed may be separated and then an organic amine is added at R.T. under N₂. In one embodiment of the present invention the organic amine may be pyridine or triethylamine.

The mixture is then cooled down to low temperature (below −10° C.) and triflic anhydride or methanesulfonic anhydride or methanesulfonyl chloride is added carefully. The reaction mixture is then usually worked-up.

In another embodiment of the present invention the solution may be added with pure seeds of the desired intermediate compound (VIII), previously prepared.

Step g stands for nucleophilic displacement of the leaving group of compounds (VIII) to give the compounds of formula (I).

In one embodiment of the present invention X may be Iodine.

In another embodiment X may be Bromine.

To a solution of intermediate compounds (VIII) in a suitable solvent which includes, but it is not limited to, a polar aprotic solvent selected in the group consisting of: dimethylformamide (DMF), dimethylsulfoxide (DMSO), N-methylpyrrolidinone (NMP), acetonitrile, a linear or branched C1-C6 alcoholic solvent or an apolar solvents, an organic acid selected in the group consisting from: methansulfonic acid, acetic acid, p-toluenesulfonic acid, trifluoroacetic acid, fumaric acid was added, followed by the addition of a halide salt with alkaline ions which includes: LiCl, LiBr, LiI, NaCl, NaBr, NaI, KCl, KBr, or KI.

The resulting mixture is usually kept at a temperature ranging from 50 to 120° C. for 2-24 hr. At the end the reaction mixture is worked-up as usual in order to provide a two layers mixture. The organic layer is usually constituted by a suitable organic solvent such as an etheral or ester solvent, as defined above.

The crude product may be used as such in the next step for the formation of the bicylic CRF antagonists which will be defined in the following

Compounds of formula (VI) may be prepared as disclosed in WO 04/062665 and WO 04/094420.

Compounds of formula (VI) may exist in the tautomeric form.

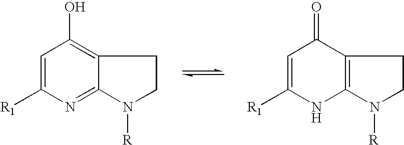

A process for the preparation of compounds (IV) is one embodiment of the present invention, starting from compounds of formula (II) and comprising the following steps according to Scheme 2:

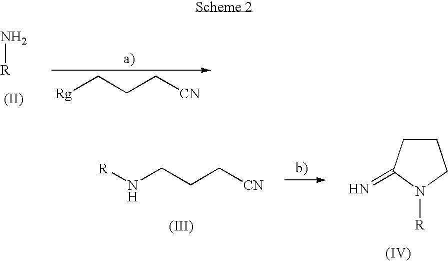

wherein R is defined as above, Rg is a reactive group selected from: halogen, reactive derivative of an alkylsulphonic acid, and
step a stands for alkylation of the suitable aryl or heteroayl amine of formula (II) with a reactive derivative of butyrronitrile in presence of a base by heating;
step b stands for the formation of the pyrrolidinone moiety of compounds (IV) which will form the cycle B present in the final compounds (I), by cyclisation of compounds (III), acid catalised and by heating to give the desired compounds (IV).

The starting R—NH₂ may be a compound generally already known in literature. If not, it may be prepared using classical approach known to the skilled person.

Step a stands for alkylation of the suitable aryl or heteroayl amine of formula (II) with a reactive derivative of butyrronitrile in presence of a base by heating.

The suitable aryl or heteroaryl amine is dissolved in a proper solvent which includes, but it is not limited to, a tertiary C1-C6 dialkylamine.

In one embodiment of the present invention the tertiary C1-C6 dialkylamine may be trietylamine or diisopropylamine together, if necessary, with a polar aprotic solvent selected in the group consisting of: dimethylformamide (DMF), dimethylsulfoxide (DMSO), N-methylpyrrolidinone (NMP), acetonitrile.

The reaction is usually conducted at a temperature comprised in the range 100-150° C. In one embodiment of the present invention the reactive derivative of butyrronitrile is an halogen derivative. In a further embodiment the halogen may be Cl or Br.

The reactive derivative is added dropwise under N₂. The reaction mixture is then stirred for 2-6 hr. The mixture is then cooled down to R.T. and diluted with a suitable solvent which includes, but it is not limited to, linear, branched or cyclic C1-C6 dialkylether.

In one embodiment of the present invention the solvent may be selected from the group consisting of: methyl-t-butyl ether, dietylether, tetrahydrofuran, or dioxane.

The reaction mixture is then worked up as usual and at the end a suitable co-solvent is added. A suitable co-solvent may be selected in the group of C1-C10 cyclic alcanes.

In one embodiment of the present invention the co-solvent may be cyclohexane.

The crude product may be used as such in the next step.

Step b stands for the formation of the pyrrolidinone moiety of compounds (I) which will form the cycle B present in the final compounds (I), by cyclisation of compounds (III).

To a suspension of intermediate compounds (III) in a suitable solvent, which includes, but it is not limited to, a linear or branched C1-C6 alcoholic solvent or a C1-C10 aromatic solvent or a linear, branched or cyclic C1-C6 dialkylether.

In one embodiment of the present invention the alcoholic solvent may be iso-propanol; the aromatic solvent may be toluene and the etheral solvent may be tetrahydrofuran (THF).

Then 1.5 eq. of an acid are added at R.T. under N₂.

The most suitable acid may be selected among the organic acid or inorganic acids common to the skilled person.

Organic acids include, but are not limited to: acetic acid, malic acid, maleic acid, fumaric acid, lactic acid, tartaric acid, citric acid, formic acid, gluconic acid, succinic acid, piruvic acid, oxalic acid, oxaloacetic acid, trifluoroacetic acid, benzoic acid, methanesulphonic acid, ethanesulphonic acid, benzenesulphonic acid, p-toluensulphonic acid, methanesulphonic acid and isethionic acid.

Inorganic acids include, but are not limited to: hydrochloric acid, hydrobromic acid, hydroiodic acid, sulphoric acid, nitric acid, phosphoric acid, hydrogen phosphoric acid.

In one embodiment of the present invention the organic acid may be p-toluenesulfonic acid or methanesulfonic acid and the inorganic acid may be hydrochloric acid (HCl).

The mixture is then usually heated to reflux for 4-8 hr, and at the end worked as usual in order to provide a two layers mixture. The organic layer is usually constituted by a suitable organic solvent which includes, but it is not limited to, chlorinated solvents or esters of organic acids.

In one embodiment of the present invention the chlorinated solvent may be dichloromethane and the ester of organic acid may be ethylacetate.

The crude product may be used as such in the next step.

In one aspect of the present invention step a and step b may be performed continuously without isolating intermediate (III), according to the following Scheme 3, in order to produce compounds of formula (IVB), which can be used as compounds (IV) after treatment in basic conditions.

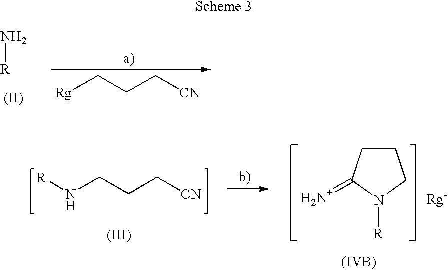

Compounds (IV) may be isolated as a suitable salt, for example hydrobromide, depending of the type of reactive butyrronitrile used in step b). Then bromobutyrronitrile will be used for obtaining compounds (IVB) as hydrobromide

The preparation of 1-[2-methyl-4-(trifluoromethyloxy)phenyl]-2-pyrrolidinimine hydrobromide included in the Experimental Section is an illustration of this alternative way of performing the process of the present invention.

A process for the preparation of compounds (VII) is another embodiment of the present invention, starting from compounds of formula (IV) and comprising the following steps:

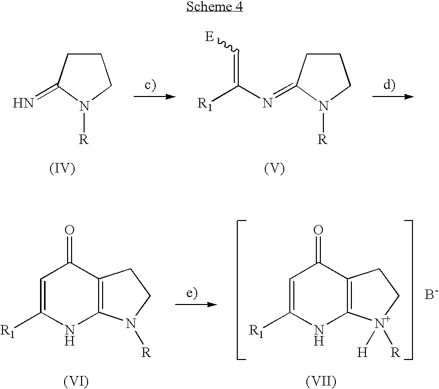

wherein R and R₁, are defined as above, and
step c stands for a Michael addition of compounds (IV) to a butynoate derivative by heating;
step d stands for cyclisation in basic conditions to give the aromatic compounds (VI);
step e stands for salt formation by addition of the suitable acid to the compounds (VI).

Compounds (IV) may be replaced in Scheme 4 by compounds (IVB) providing an initial step c′ of basic treatment in a suitable base as illustrated in the following Scheme 5.

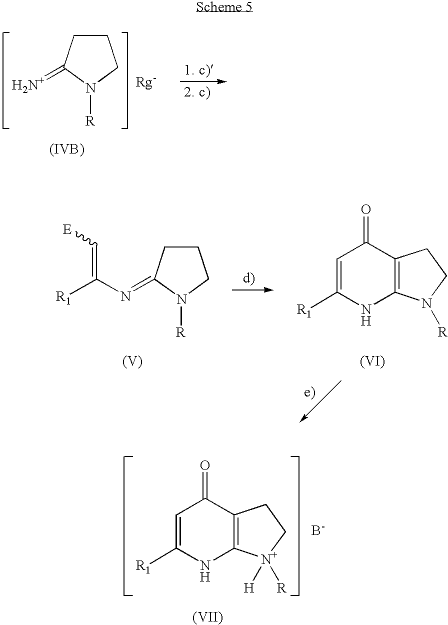

Step c stands for a Michael addition of intermediate compounds (IV) to a suitable butynoate derivative.

To a solution of intermediate compounds (IV) in a suitable solvent which includes but it is not limited to, an etheral solvent, a polar aprotic solvent or an alcoholic solvent as defined above, 1.0-1.5 eq of an ester derivative of 2-butynoate is added at R.T. under N₂.

In one embodiment of the present invention the ester derivative of 2-butynoate may be ethyl 2-butynoate.

The mixture was heated to reflux and kept for 2-20 hr before allowing cooling down to R.T. The reaction mixture was then evaporated to dryness. The crude oil may be used as such in the next step.

Step d stands for cyclisation in basic conditions of the intermediate compounds (V) to give the aromatic compounds (VI). To a solution of the intermediate compounds (V) in a suitable solvent selected among etheral solvents, alcoholic solvents or polar aprotics solvents as defined above, a suitable base selected in the group consisting from: potassium t-butoxide, lithium hexamethyldisilazane, diazabicyclo[2.2.2]octane, 1,8-diazabicyclo[5.4.0]undecen-7-ene, sodium hydride; is added at R.T. under N₂.

The reaction mixture is then generally heated to reflux and stirred for 2-14 hr and at the end worked as usual in order to provide a two layers mixture. The organic layer is usually constituted by a suitable organic solvent which includes, but it is not limited to, chlorinated solvents.

In one embodiment of the present invention the chlorinated solvent may be dichloromethane.

The crude product may be used as such in the next step.

Step e stands for the formation of compounds (VII) by addition of the suitable acid to the intermediate compounds (VI).

A compound (VI) is dissolved in a suitable solvent which includes, but it is not limited to, a linear, branched or cyclic C1-C6 dialkylether, a linear or branched aliphatic C1-C6 ketonic solvent. The solution is then treated with a suitable inorganic acid.

In one embodiment of the present invention the ketonic solvent may be acetone or 2-butanone, the etheral solvent may be tethrahydrofurane (THF) and the acid may be a sulphonic acid. In a further embodiment the sulphonic acid may be p-toluensulphonic acid or methanesulphonic acid.

In another embodiment the solution may be added with pure seeds of the desired intermediate compound (VII), previously prepared.

After 2-10 hr the suspension is filtered and the cake washed with another solvent.

The collected solid is then dried in the usual way.

We have found, and it is another embodiment of the present invention, that the formation of compounds (VII) improves the process management as far as regards the purification procedures. In fact, by the introduction of these salts formation it is now possible to have reasonable clean intermediates without using chromatographic procedures. In addition isolation of such intermediates allows a better control over the impurity profile in the next steps.

### EXAMPLES

In the Intermediates and Examples unless otherwise stated:

All temperatures refers to ° C. Infrared spectra were measured on a FT-IR instrument. Compounds were analysed by direct infusion of the sample dissolved in acetonitrile into a mass spectra operated in positive electro spray (ES⁺) ionisation mode. Proton Magnetic Resonance (¹H-NMR) spectra were recorded at 400 MHz, chemical shifts are reported in ppm downfield (d) from Me₄Si, used as internal standard, and are assigned as singlets (s), broad singlets (bs), doublets (d), doublets of doublets (dd), triplets (t), quartets (q) or multiplets (m). A strategy comprising of NOE (Nuclear Overhauser Effect) correlation and/or 1H,15N long range scalar correlations measurements has been implemented in order to allow elucidation of possible regio-isomers structure of compounds of the present invention. Proposed structures were verified by measurement of the vicinity in the space of key hydrogens, thus 1 D Nuclear Overhauser difference spectra were used to measure 1H,1H-dipole-dipole correlations.

In cases where NOE measurements were not conclusive, 1H,15N long range scalar correlations were measured via 1H,15N-HMBC experiments. A delay corresponding to an average long range scalar coupling 2,3J(1H,15N) of 6 Hz was set for optimal result.

Column chromathography was carried out over silica gel (Merck AG Darmstaadt, Germany). The following abbreviations are used in the text: EtOAc=ethyl acetate, cHex=cyclohexane, CH₂Cl₂=DCM, dichloromethane, Et₂O=dietyl ether, DMF=N,N′-dimethylformamide, DIPEA=N,N-diisopropylethylamine, MeOH=methanol, Et₃N=triethylamine, TFA=trifluoroacetic acid, THF=tetrahydrofuran, NMP=N-methyl-2-pyrrolidinone, MTBE=methyl-tert-butyl ether, IPA=isopropanol, DABCO=Diazabicyclo[2.2.2]octane, DBU=1,8-Diazabicyclo[5.4.0]undecen-7-ene, BINAP=2,2′-Bis(diphenylphosphino)-1,1′-binaphthyl, Acac=2,4-Pentanedione, MEK=methyl ethyl ketone.

The method HPLC used for the purity determination is the following:

| Column | Phenomenex Luna 3μ C18(2) - 50 × 2.0 mm | |
|---|---|---|
| Wavelength | 220 nm | |
| Flow | 1 mL | |
| Injection volume | 5 uL (2 uL) | |
| Temperature | 40° C. | |
| Run Time | 10 min | |
| Sample conc. | ca. 0.5 mg/mL (ca. 1 mg/mL) | |
| Mobile Phase | Solution A: 0.05% TFA in water | |
| | Solution B: 0.05% TFA in ACN | |
| Gradient | FAST gradient: | |
| | 0.00-8.00 min: | from 100% A down to 5% |
| | 8.01-8.10 min: | from 5% A up to 100% |
| | 8.11-10.00 min: | 100% of A |

### Example 1

### Preparation of Intermediate (III)

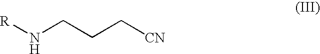

A solution of tertiary amines (e.g. TEA, DIPEA; 1 eq) and RNH₂ (1 eq.) in polar aprotic solvent (e.g. DMF, NMP) was heated to 100-150° C. 4-X-butyrronitrile, where X=Cl or Br; 1 eq) was added dropwise under N₂. The reaction mixture was heated for 2-6 hr. The mixture was cooled down to R.T. and diluted with ether (e.g. MTBE, Et₂O). Water was added and the phases were separated. The organic layer was further washed with water and evaporated to low volume. New ether was added and the mixture again evaporated to low volume. The mixture was treated with cyclic alcanes (e.g cyclohexane) over 20 minutes and the resulting suspension aged at room temperature for 1-5 hr. The suspension was filtered and the cake washed with a mixture ether/alcane mixture. The title compound was collected as a solid.

### 4-{[2-methyl-4-(methyloxy)phenyl]amino}butanenitrile

Yield: 65-70% th

### 4-{[2-methyl-6-(methyloxy)-3-pyridinyl]amino}butanenitrile

Yield: 80%

All the analytical data are set forth in the following Table 1-1

TABLE 1-1

| Cpd. | | |
|---|---|---|
| No. | R | Analytical Data |
| 1-1 | 2-methyl-4-methoxy- | NMR (¹H, CDCl₃): NMR (¹H, |
| | phenyl | DMSO-d₆): δ 6.65 (d, 1H), 6.63 |
| | | (dd, 1H), 6.47 (d, 1H), 4.49 (bt, |
| | | 1H), 3.64 (s, 3H), 3.10 (q, 2H), |
| | | 2.59 (t, 2H), 2.09 (s, 3H), 1.86 |
| | | (m, 2H). |
| | | M/S (m/z): 205 [MH]⁺ |
| | | HPLC % a/a > 99 |
| 1-2 | 2-methyl-4-methoxy- | NMR (¹H, DMSO-d₆): δ 6.94 (d, |
| | 3-piridynyl | 1H), 6.53 (d, 1H), 3.85 (s, 3H), |
| | | 3.28 (t, 2H), 3.14 (bs, 1H), 2.50 |
| | | (t, 2H), 2.33 (s, 3H), 1.97 (m, |
| | | 2H) |
| | | M/S (m/z): 206 [MH]⁺ |
| | | HPLC % a/a 81% |

### Example 2

### Preparation of Intermediates (IV)

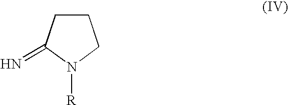

To a suspension of intermediate (III) in alcoholic solvents (e.g IPA), aromatic solvents (e.g. Toluene) or etheral solvents (e.g THF), an organic acid (e.g. p-toluenesulfonic acid; methanesulfonic acid) or a mineral acid (e.g. HCl 5-6N in IPA) (1.5 eq) was added at R.T. under N₂. The mixture was heated to reflux for 4-8 hr, allowed to cool down to R.T. and evaporated to low volume. Water was added, the clear solution evaporated again to low volume and treated with NaOH aqueous solution. The mixture was extracted with organic solvent (DCM, ethyl acetate) and the organic layer further washed with NaCl aqueous solution. The organic layer was evaporated down to dryness. The crude product was used as such in the next step.

### 1-[2-methyl-4-(methyloxy)phenyl]-2-pyrrolidinimine

4-{[2-Methyl-4-(methyloxy)phenyl]amino}butanenitrile (0.78 Kg) was treated with HCl 10% in water (2.34 L) and the solution heated to 85° C. After 4 hour the mixture was cooled down to 20° C., diluted with NaOH 10% and extracted with DCM. The aqueous layer was further extracted with DCM. The combined organic layers were washed with NaCl 15%. The collected organic phase was diluted with THF, distilled to about 1 L volume (50° C. jacket, 250 mbar). THF was added and the mixture distilled again to about 1 L. Fresh THF was added one more time and the mixture again distilled down to about 4 L. The product is used as such in the next step.

Yield: 95-99% th

### 1-[2-methyl-6-(methyloxy)-3-pyridinyl]-2-pyrrolidinimine

Yield: 78% th

All the analytical data are set forth in the following Table 2-1

TABLE 2-1

| Cpd. | | |
|---|---|---|
| No. | R | Analytical Data |
| 2-1 | 2-methyl-4-methoxy- | NMR (¹H, DMSO-d₆): δ 7.09 (d, |
| | phenyl | 1H), 6.87 (d, 1H), 6.80 (dd, 1H), |
| | | 5.8-5.4 (b, 1H), 3.75 (s, 1H), |
| | | 3.54 (t, 2H), 2.51 (t, 2H), 2.11 |
| | | (s, 3H), 2.01 (m, 2H). |
| | | M/S (m/z): 205 [MH]⁺ |
| | | HPLC % a/a > 98 |
| 2-2 | 2-methyl-4-methoxy- | NMR (¹H, DMSO-d₆): δ 7.53 (d, |
| | 3-piridynyl | 1H), 6.68 (d, 1H), 6.1-5.8 (b, |
| | | 1H), 3.84 (s, 3H), 3.55 (t, 2H), |
| | | 2.50 (m, 2H), 2.26 (s, 3H), 2.02 |
| | | (m, 2H) |
| | | M/S (m/z): 205 [MH]⁺ |
| | | HPLC % a/a 86% |
| 2-3 | 2-methyl-4- | NMR (¹H, CDCl₃): δ 7.84 (d, |
| | trifluormethoxy- | 1H), 7.30 (d, 1H), 7.12 (s, 1H), |
| | phenyl | 7.09 (d, 1H), 6.98 (d, 1H), 6.63 |
| | | (s, 1H), 4.68 (s, 1H), 4.10 (t, |
| | | 2H), 3.91 (t, 2H), 3.63 (t, 2H), |
| | | 3.50 (t, 2H), 2.36 (s, 3H), 2.29 |
| | | (s, 3H). |
| | | MS (m/z): 459 [MH]⁺ |

### Example 3

### Preparation of Compounds (IVB)

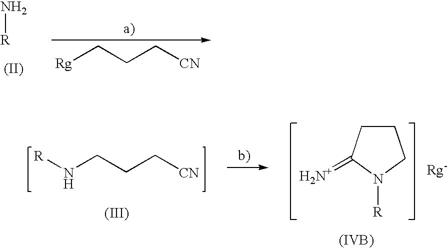

### 1-[2-methyl-4-(trifluoromethyloxy)phenyl]-2-pyrrolidinimine hydrobromide

2-Methyl-4-trifluoromethyloxyaniline (30 g) was dissolved in NMP (90 ml). The resulting solution was heated up to 100° C. Neat bromobutyrronitrile (1.1 eq; 17.2 mL) was then added and the resulting solution was heated at 115-118° C. for 2-4 hours.

The reaction was then allowed to cool to 45° C. in 30 min. A seed of the desired compound (0.03 g) was added. MTBE (270 ml) was added at 45° C. in 30-40 min. The resulting suspension was cooled to 20° C. in 20 min, stirred for 2 hrs and then was filtered. The cake was washed with a mixture of 3:1 MTBE/NMP (3×60 mL) and the solid dried overnight at 70° C. for 6 hrs.

Yield: 88% th from 2-Methyl-4-trifluoromethyloxyaniline

NMR (1H, DMSO-d6): 9.83 (s, 1H), 8.62 (s, 1H), 7.58 (d, 1H), 7.48 (d, 1H), 7.41 (dd, 1H), 3.92 (t, 2H), 3.08 (m, 2H), 2.24 (m, 2H), 2.24 (s, 3H).

HPLC % a/a 99%

### Example 4

### Preparation of Intermediates (V)

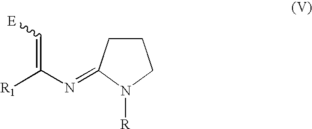

To a solution of intermediate (IV) in etheral solvent (e.g. THF), polar aprotic solvents (e.g. acetonitrile), or alcoholic solvents (e.g. IPA); ethyl-2-butynoate (1.0-1.5 eq) was added at R.T. under N₂. The mixture was heated to reflux and aged for 2-20 hr before allowing cooling down to R.T. The reaction mixture was evaporated to dryness. The crude oil was used as such in the next step.

### Ethyl-3-({(2E)-1-[2-methyl-4-(methyloxy)phenyl]-2-pyrrolidinylidene}amino)-2-butenoate

The solution containing 1-[2-methyl-4-(methyloxy)phenyl]-2-pyrrolidinimine, as previously prepared, was treated with ethyl-2-butynoate (0.49 L). The mixture was heated to reflux for 12-14 hours. The mixture was allowed to cool to room temperature. The product is used as such in the next step.

Yield 80-90% th

### Ethyl-3-({2E)-1-[2-methyl-6-(methyloxy)-3-pyridinyl]-2-pyrrolidinylidene}amino)-2-butenoate

Yield 89% th

### Ethyl-3-({(2E)-1-[2-methyl-4-(trifluoromethyloxy)phenyl]-2-pyrrolidinylidene}amino)-2-butenoate

1-[2-methyl-4-(trifluoromethyloxy)phenyl]-2-pyrrolidinimine hydrobromide (1.4 kg) was treated with 10% NaOH acq. solution (4.2 L) and extracted with DCM (4.2 L). The aqueous layer was further extracted with DCM (2.8 L). The combined organic layers were washed with aqueous sodium chloride w/v 15% (5.6 L). The collected organic phase was diluted with toluene (7 L), distilled to 2.8 L, diluted with toluene (14 L) and distilled to 2.8 L. The solution was treated with ethyl-2-butynoate (1.1 eq, 0.53 L). The mixture was heated to reflux for about 9 hours. The mixture was allowed to cool to room temperature. The product is used as such in the next step.

All the analytical data are set forth in the following Table 3-1

TABLE 3-1

| Cpd. | | |
|---|---|---|
| No. | R | Analytical Data |
| 3-1 | 2-methyl-4-methoxy- | M/S (m/z): 317 [MH]⁺ |
| | phenyl | HPLC % a/a > 90 |
| 3-2 | 2-methyl-4-methoxy- | M/S (m/z): 318 [MH]⁺ |
| | 3-piridynyl | HPLC % a/a 79% |

### Example 5

### Preparation of Intermediates (VI)

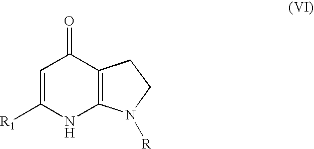

To a solution of intermediate (V) in etheral solvents (e.g THF), alcoholic solvents (e.g. IPA), polar aprotics solvents (e.g. acetonitrile, DMF) was added at R.T. under N₂, a base (e.g. t-BuOK, LiHMDS, DABCO, DBU, NaH). The reaction mixture was heated to reflux and stirred for 2-14 hr. The solution was allowed to cool down to R.T., evaporated to low volume and diluted with chlorinated solvent (e.g. DCM). The organic layer was washed with sat.aq. NH₄Cl; followed by NaCl aqueous solution. The organic layer was evaporated to dryness and the crude product was used as such in the next step.

### 6-methyl-1-[2-methyl-4-(methyloxy)phenyl]-1,2,3,7-tetrahydro-4H-pyrrolo[2,3-b]pyridin-4-one

The solution from the previous step containing ethyl-3-({(2E)-1-[2-methyl-4-(methyloxy)phenyl]-2-pyrrolidinylidene}amino)-2-butenoate was treated with t-BuOK 1M in THF (7.8 L; prepared by dissolution of solid tBuOK-2 eq- in THF). The t-BuOK solution was added the first 20% in 30 minutes and the remaining part in 40-50 minutes. The mixture was refluxed for 6 hours. Then it was cooled to 20° C., concentrated (50° C. jacket, 300-250 mbar), diluted with NH₄Cl sat. sol. and extracted with DCM. The aqueous layer was back extracted with DCM. The combined organic phases were washed with NaCl 15%. The organic layer was distilled down to about 1 L, diluted with MEK and evaporated down to about 4 L. Fresh MEK was added and the mixture was concentrated down to about 4 L. The product is used as such in the next step.

Yield 75-85% th

### 6-methyl-1-[2-methyl-6-(methyloxy)-3-pyridinyl]-1,2,3,7-tetrahydro-4H-pyrrolo[2,3-b]pyridin-4-one

Yield: 15-20% th

### 6-methyl-1-[2-methyl-4-(trifluoromethyloxy)phenyl]-1,2,3,7-tetrahydro-4H-pyrrolo[2,3-b]pyridin-4-one

The solution from the previous step containing ethyl-3-({(2E)-1-[2-methyl-4-(trifluoromethyloxy)phenyl]-2-pyrrolidinylidene}amino)-2-butenoate was treated with t-BuOK 1M in THF (8.26 L; prepared by dissolution of solid tBuOK-2 eq- in THF). The t-BuOK solution was added in 30 minutes. The mixture was refluxed for 3 hours. Then it was cooled to 20° C., concentrated to 4.2 L (50° C. jacket, 300-250 mbar), diluted with NH₄Cl sat. sol. (7 L) and extracted with DCM (11.2 L).The aqueous layer was back extracted with DCM (4.2 L).The combined organic phases were washed with NaCl 15% (2.8 L). The organic layer was distilled down to 2.8 L (50° C. jacket, 300 mbar), diluted with THF (11.2 L) and evaporated down to 2.8 L. Fresh THF (7 L) was added. The solution was treated with CH₃SO₃H (0.28 L) in a dropwise fashion over 1 hr. Precipitation occurred during the addition of the acid. The suspension was aged for 4-6 hours, then filtered and the cake washed with THF (5.6 L). The collected solid was placed in the oven at 70° C., under reduced pressure for at least 5-6 hours.

Overall yield: 50-65%

All the analytical data are set forth in the following Table 4-1

TABLE 4-1

| Cpd. | | |
|---|---|---|
| No. | R | Analytical Data |
| 4-1 | 2-methyl-4-methoxy- | NMR (¹H, DMSO-d₆): δ 9.8 (b, |
| | phenyl | 1H), 7.08 (d, 1H), 6.80 (d, 1H), |
| | | 6.75 (dd, 1H), 5.92 (s, 1H), 3.72 |
| | | (s, 3H), 3.68 (t, 2H), 2.89 (t, |
| | | 2H), 2.12 (s, 3H), 2.02 (s, 3H). |
| | | M/S (m/z): 271 [MH]⁺ |
| | | HPLC % a/a 75-80 |
| 4-2 | 2-methyl-4-methoxy- | NMR (¹H, DMSO-d₆): δ 9.93 |
| | 3-piridynyl | (bs, 1H), 7.55 (d, 1H), 6.67 (d, |
| | | 1H), 5.99 (s, 1H), 3.84 (s, 3H), |
| | | 3.73 (t, 2H), 2.94 (t, 2H), 2.28- |
| | | 2.07 (2s, 6H). |
| | | M/S (m/z): 272 [MH]⁺ |
| | | HPLC % a/a 35-40 |

### Example 6

### Preparation of Intermediates (VII)

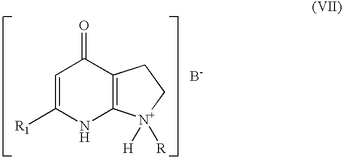

Intermediate (VI) was dissolved in etheral solvents (e.g THF), ketonic solvents (e.g acetone, 2-butanone), treated with sulphonic acid (e.g p-toluensulphonic; methanesulphonic, triflic anhydride) and seeded with intermediate (VII). After 2-10 hr the suspension was filtered and the cake washed with further solvent. The collected solid was placed in the oven at 40° C. under reduced pressure for 10-24 hr.

### 6-methyl-1-[2-methyl-4-(methyloxy)phenyl]-1,2,3,7-tetrahydro-4H-pyrrolo[2,3-b]pyridin-4-one methanesulfonate

The solution containing 6-methyl-1-[2-methyl-4-(methyloxy)phenyl]-1,2,3,7-tetrahydro-4H-pyrrolo[2,3-b]pyridin-4-one as previously prepared, was treated with CH₃SO₃H (0.187 L) in a dropwise fashion over 20-25 minutes (temperature rose from 20° C. to 30° C. internally) and seeded with the title compound. Precipitation occurred soon after seeding. The suspension was aged for 6 hours, then filtered and the cake washed with 2-butanone. The collected solid was placed in the oven at 40° C., under reduced pressure for 10-12 hours.

Yield: 90-95% th

### 6-methyl-1-[2-methyl-4-(methyloxy)phenyl]-1,2,3,7-tetrahydro-4H-pyrrolo[2,3-b]pyridin-4-one 4-methylbenzenesulfonate

Yield: 54% th

### 6-methyl-1-[2-methyl-4-(trifluoromethyloxy)phenyl]-1,2,3,7-tetrahydro-4H-pyrrolo[2,3-b]pyridin-4-one trifluoromethanesulfonate

A saturated aqueous solution of NaHCO₃ (6 L) was added at room temperature to a suspension of 6-methyl-1-[2-methyl-4-(trifluoromethyloxy)phenyl]-1,2,3,7-tetrahydro-4H-pyrrolo[2,3-b]pyridin-4-one (1 Kg) in dichloromethane (10 L). The resulting mixture was stirred for 20 min at room temperature. The separated organic phase was washed with a 15% (w/v) aqueous solution of NaCl (3 L), then was diluted with CH₂Cl₂ (10 L). The resulting solution was distilled down to 10 L. Fresh CH₂Cl₂ (5 L) was added and the solution was concentrated to 10 L. Fresh CH₂Cl₂ (5 L) was added and the solution was concentrated again to 10 L. The solution as such is used in the next step.

All the analytical data are set forth in the following Table 5-1

TABLE 5-1

| Cpd. | | |
|---|---|---|
| No. | R | Analytical Data |
| 5-1 | 2-methyl-4-methoxy- | NMR (¹H, DMSO-d₆): δ 12.36- |
| | phenyl | 12.70 (2bs, 2H), 7.33 (d, 1H), |
| | | 7.00 (d, 1H), 6.92 (dd, 1H), |
| | | 6.26 (s, 1H), 4.02 (bm, 2H), |
| | | 3.81 (s, 3H), 3.11 (m, 2H), 2.34 |
| | | (s, 3H), 2.25-2.20 (2s, 6H). |
| | | HPLC a/a > 98 |
| 5-2 | 2-methyl-4-methoxy- | NMR (¹H, DMSO-d₆): δ 12.34- |
| | 3-piridynyl | 12.00 (2bs, 2H), 7.50 (d, 2H), |
| | | 7.32 (d, 1H), 7.13 (d, 2H), 7.00 |
| | | (d, 1H), 6.92 (dd, 1H), 6.26 (s, |
| | | 1H), 4.02 (m, 2H), 3.80 (s, 3H), |
| | | 3.11 (m, 2H), 2.30 (s, 3H), |
| | | 2.24-2.19 (2s, 6H). |
| | | HPLC a/a > 98 |
| 5-3 | 2-methyl-4- | NMR (1H, DMSO-d6): 12.48 |
| | trifluoromethoxy | (s, 1H), 12.06 (s, 1H), 7.56 (d, |
| | | 1H), 7.48(d, 1H), 7.36 (dd, 1H), |
| | | 6.30 (s, 1H), 4.04 (s, 2H), 3.11 |
| | | (t, 2H), 2.29 (s, 3H), 2.26(s, |
| | | 6H). |
| | | HPLC % a/a 98% |

### Example 7

### Preparation of Intermediates (VIII)

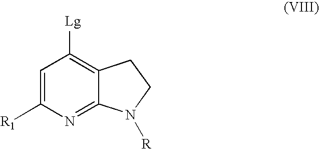

To a suspension of intermediate (VII) in chlorinated solvents (e.g. DCM), an inorganic base in aqueous solution was added. After phase separation the organic one was washed with NaCl aqueous solution and dried. An amine (e.g. pyridine, TEA) was added at R.T. under N₂ at the organic solution. The mixture was cooled down to low temperature (below −10° C.) and triflic anhydride or methanesulfonic anhydride or methanesulfonyl chloride was added in a dropwise fashion. The reaction mixture was allowed to warm up to 5° C. over 30 minutes and treated with sat.aq NaHCO₃. Phases were separated and the organic one was further washed with water and concentrated to oil. The oil was dissolved in alcoholic solvent (IPA) and seeded with intermediate (VIII). The suspension was stirred for 1-4 hr, then water was added over 30 minutes and the mixture aged for additional 1-5 hr. The suspension was filtered, the cake washed with an alcohol/water mixture 1/1, collected and dried in the oven at 35-40° C. under high vacuum for 12-14 hours. The title compound was obtained as a solid.

### 6-methyl-1-[2-methyl-4-(methyloxy)phenyl]-2,3-dihydro-1H-pyrrolo[2,3-b]pyridin-4-yl trifluoromethanesulfonate

6-Methyl-1-[2-methyl-4-(methyloxy)phenyl]-1,2,3,7-tetrahydro-4H-pyrrolo[2,3-b]pyridin-4-one methanesulfonate as previously prepared (0.4 Kg; 1 eq) was suspended in DCM (4 L) and treated with NaHCO₃ sat. sol. (2.4 L). Phases were allowed to separate and the organic one washed with NaCl 15%. The organic layer was diluted with DCM and the solution distilled down to 4 L. Fresh DCM added again and the mixture distilled down to 4 residual litres. The solution was treated with Pyridine (0.097 L, 1.1 eq) and cooled down to −15° C. Triflic anhydride (0.193 L, 1.05 eq) was added over 60 min keeping the temperature below −10° C. The mixture was allowed to warm up to 5° C. over 20 min and quenched with NaHCO₃ sat. over 20 minutes keeping temperature at 5° C. The biphasic mixture was allowed to warm up to R.T. while stirring for additional 20 minutes to complete CO₂ evolution; then allowed to separate. The organic one further washed with water, distilled down to 1.6 L (50° C. jacket, 250 mbar) and diluted with IPA. The solution was distilled down to about 2 L (50° C. jacket, 100-150 mbar), diluted with fresh IPA and again distilled down to about 2 L (50° C. jacket, 100-150 mbar). The solution was brought to room temperature and seeded with the title compound. The slurry was aged for 60 min. Water was added over 30 min and the resulting suspension aged for 90 min before being filtered. The cake was washed with IPA-water 1:1, collected and placed in the oven at 35° C., under reduced pressure overnight.

Yield: 80-85% th

### 6-methyl-1-[2-methyl-4-(methyloxy)phenyl]-2,3-dihydro-1H-pyrrolo[2,3-b]pyridin-4-yl methanesulfonate

Yield: 82% th

### 6-methyl-1-[2-methyl-4-(trifluoromethyloxy)phenyl]-2,3-dihydro-1H-pyrrolo[2,3-b]pyridin-4-yl trifluoromethanesulfonate

Pyridine (1.1 equiv, 0.21 L) was added to the solution containing 6-methyl-1-[2-methyl-4-(trifluoromethyloxy)phenyl]-1,2,3,7-tetrahydro-4H-pyrrolo[2,3-b]pyridin-4-one trifluoromethanesulfonate and the resulting mixture was cooled down to −15° C. Neat trifluoromethanesulfonic anhydride (1.05 equiv, 0.41 L) was then added dropwise maintaining, the temperature ranging below −10° C., then the solution was heated up to 5° C. in 40 min. A saturated aqueous solution of NaHCO₃ (5 L) was then added dropwise in 30 min, keeping the temperature below 5° C. The solution was finally heated up to 20° C. in 30 min. The separated organic layer was then washed with water (5 L) and concentrated to 4 L. Fresh IPA (8 L) was then added and the resulting solution was distilled down to 8 L. Fresh IPA (8 L) was added and the solution was distilled down to 8 L. The solution was cooled down to room temperature. A yellow solid precipitated at room temperature. The resulting suspension was stirred for 0.5 h at room temperature, then water (8 L) was added and the suspension was stirred overnight, filtered and the solid was washed with a mixture of IPA/water 1:1 (2×2 L) and dried overnight at 40° C. under high vacuum.

Overall yield: 80-95%

All the analytical data are set forth in the following Table 6-1

TABLE 6-1

| Cpd. | | |
|---|---|---|
| No. | R | Analytical Data |
| 6-1 | 2-methyl-4-methoxy- | NMR (¹H, DMSO-d₆): δ 7.17 (d, |
| | phenyl | 1H), 6.85 (d, 1H), 6.77 (dd, 1H), |
| | | 6.40 (s, 1H), 3.89 (t, 2H), 3.73 |
| | | (s, 3H), 3.16 (t, 2H), 2.17-2.11 |
| | | (2s, 6H) |
| | | M/S (m/z): 403 [MH]⁺ |
| | | HPLC % a/a > 99 |
| 6-2 | 2-methyl-4-methoxy- | NMR (¹H, DMSO-d₆): δ 7.22 (d, |
| | 3-piridynyl | 1H), 6.89 (d, 1H), 6.82 (dd, 1H), |
| | | 6.49 (s, 1H), 3.91 (t, 2H), 3.76 |
| | | (s, 3H), 3.52 (s, 3H), 3.18 (t, |
| | | 2H), 2.20 (s, 3H), 2.15 (s, 3H) |
| | | M/S (m/z): 349 [MH]⁺ |
| | | HPLC % a/a > 99 |
| 6-3 | 2-methyl-4-trifluoro- | NMR (1H, DMSO-d6): 7.44 (d, |
| | methoxyphenyl | 1H), 7.34 (s, 1H), 7.24 (d, 1H), |
| | | 6.52 (s, 1H), 3.99 (t, 2H), 3.22 |
| | | (t, 2H), 2.23 (s, 3H), 2.21 (s, |
| | | 3H). |
| | | HPLC a/a 98% |

### Example 8

### General Preparation of Compounds of Formula (I)

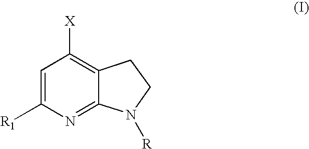

To a solution of intermediate (VIII) in polar aprotic solvents (e.g. DMF, NMP, acetonitrile), alcoholic solvents (e.g. IPA) or apolar solvents (e.g. toluene) was added an organic acid (e.g. methansulfonic acid, acetic acid, p-toluenesulfonic acid, trifluoroacetic acid, fumaric acid) followed by a halide salt (e.g. LiX, NaX, KX; X=Cl, Br, I) and the resulting mixture was heated at 50-120° C. for 2-24 hr.

The mixture was allowed to cool down to R.T. and diluted with ethereal or estereal solvents (e.g. MTBE, AcOEt) and washed with NaOH 1 N; the organic phase was washed twice with water then dried over Na₂SO₄. The removal of solvents under reduced pressure gave the intermediate (VIII) that was used as such in the next step.

### 3-Chloro-6-methyl-1-[2-methyl-4-(methyloxy)phenyl]-2,3-dihydro-1H-pyrrolo[2,3-b]pyridine

Yield: 85-95% th

### 3-Bromo-6-methyl-1-[2-methyl-4-(methyloxy)phenyl]-2,3-dihydro-1H-pyrrolo[2,3-b]pyridine

To a solution of 6-methyl-1-[2-methyl-4-(methyloxy)phenyl]-2,3-dihydro-1H-pyrrolo[2,3-b]pyridin-4-yl trifluoromethanesulfonate as previously prepared (1.2 Kg, 1.0 eq) in DMF (4.2 L) under a N₂ atmosphere, CH₃SO₃H (232.25 mL) was added followed by sodium bromide (460.33 g). The resulting mixture was heated at 85° C. for 2.5 h.

The mixture was diluted with MTBE and washed with NaOH 1 N; the aqueous phase was extracted again with MTBE and the combined organic phases washed twice with water. The organic layer was distilled down to 3.0 L (50° C. jacket, 500 mbar), diluted with fresh DMF and again distilled down to 3.0 L (50° C. jacket, 100-150 mbar). The DMF solution was used as such in the next step.

Yield 85-95% th

### 3-Iodo-6-methyl-1-[2-methyl-4-(methyloxy)phenyl]-2,3-dihydro-1H-pyrrolo[2,3-b]pyridine

To a solution of 6-methyl-1-[2-methyl-4-(methyloxy)phenyl]-2,3-dihydro-1H-pyrrolo[2,3-b]pyridin-4-yl trifluoromethanesulfonate (300 g, 1.0 eq.) in NMP (1.05 L) under a N₂ atmosphere, CH₃SO₃H (58.06 mL) was added followed by potassium iodide (185.7 g). The resulting mixture was heated at 85° C. for 7 h.

The mixture was diluted with AcOEt and washed with NaOH 1 N; the organic phases washed twice with water. The organic layer was distilled down to about 1 L (50° C. jacket, 500 mbar), diluted with fresh NMP and again distilled down to about 1 L (50° C. jacket, 100-150 mbar). The NMP solution was used as such in the next step. The HPLC purity was greater then 92% a/a.

Yield: 85-95% th

### 4-Iodo-6-methyl-1-[2-methyl-6-(methyloxy)-3-pyridinyl]-2,3-dihydro-1H-pyrrolo[2,3-b]pyridine

The title compound may be prepared according to the procedure described above.

### 3-Iodo-6-methyl-1-[2-methyl-4-(trifluoromethyloxy)phenyl]-2,3-dihydro-1H-pyrrolo[2,3-b]pyridine

To a solution of 6-methyl-1-[2-methyl-4-(trifluoromethyloxy)phenyl]-2,3-dihydro-1H-pyrrolo[2,3-b]pyridin-4-yl trifluoromethanesulfonate (0.4 kg) in NMP (1.6 L) under a N₂ atmosphere, CH₃SO₃H (0.068 L, 1.2 equiv) was added followed by potassium iodide (2.0 eq, 0.291 kg). The resulting mixture was heated to 90° C. for 2 hrs.

The mixture was cooled down to 25° C., diluted with AcOEt (4 L) and washed with NaOH 1 N (2 L) to reach pH=8-9, then the organic layer was washed 2 times with water (1.6 L). The organic layer was distilled down to 1.2 L, further ethyl acetate was added (2 L) diluted and the mixture was distilled down to 1.2 L. NMP (0.8 L) was added and again distilled down to 1.2 L. The NMP solution was used s such in the next step.

The HPLC purity was greater then 95% a/a.

All the analytical data are set forth in the following Table 7-1

TABLE 7-1

| Cpd. | | | |
|---|---|---|---|
| No. | X | R | Analytical Data |
| 7-1 | Cl | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.43 (s, 1H), 3.86 (t, 2H), 3.76 |
| | | | (s, 3H), 3.11 (t, 2H), 2.14 (2s, |
| | | | 6H) |
| | | | M/S (m/z): 289/291 [MH]⁺ |
| | | | HPLC % a/a > 92% |
| 7-2 | Br | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.56 (s, 1H), 3.86 (t, 2H), 3.76 |
| | | | (s, 3H), 3.06 (t, 2H), 2.15-2.14 |
| | | | (2s, 6H) |
| | | | M/S (m/z): 333/335 [MH]⁺ |
| | | | HPLC % a/a > 90% |
| 7-3 | I | 2-methyl-4-methoxy- | NMR (¹H, DMSO-d₆): δ 7.17 (d, |
| | | phenyl | 1H), 6.87 (d, 1H), 6.80 (dd, 1H), |
| | | | 6.56 (s, 1H), 3.86 (t, 2H), 3.76 |
| | | | (s, 3H), 3.06 (t, 2H), 2.15-2.14 |
| | | | (2s, 6H) |
| | | | M/S (m/z): 381 [MH]⁺ |
| | | | HPLC % a/a > 90% |

### Example 9

### General Preparation of Compounds of Formula (IA)

A solution of a copper catalyst (e.g. CuI, CuBr, Cu₂Br, Cu(AcO)₂, Cu₂O) and a ligand (e.g. cis- or trans-N,N′-dimethyl-1,2-cyclohexanediamine, a mixture of cis- and trans-N,N′-dimethyl-1,2-cyclohexanediamine, cis- or trans-1,2-cyclohexanediamine, a mixture of cis- and trans-1,2-cyclohexanediamine, N,N′-dimethyl-1,2-diaminoethane, NN,N′N′-tetramethyl-1,2-diaminoethane, ethanolamine, 1,10-phenantroline, PPh₃, BINAP, Acac) was prepared in a suitable solvent (e.g. DMF, NMP, DMSO, acetonitrile, dioxane, toluene).

Then an inorganic or organic base (e.g. potassium carbonate, cesium carbonate, potassium phosphate, ter-BuOK, DBU, TEA, DIPEA) was added followed by the Z-W-reactive derivative and the intermediate (VIII). The resulting mixture was heated at 80°-150° C. for 4-48 hr.

The mixture was cooled at 60° C. and water was added dropwise. The suspension was stirred at room temperature for 1 hr, then the white precipitate was filtered and washed upon the filter once with a 1/2 mixture of DMF/water, then twice with water. The solid was dried at 80° C. for 24 hr to obtain the title compound as crude. The crude was dissolved at room temperature in a suitable mixture, such as DCM/MeOH 9/1. The solution was filtered through a carbon pad washing upon the filter with a DCM/MeOH mixture 9/1. The mixture underwent a solvent exchange into a suitable solvent such as alcohols (e.g. Methanol) or aromatic ether (e.g. Anisole). The resulting suspension was aged for 2 hr, filtered and washed upon the filter with MeOH. The collected solid was dried at 80° C. for 24 hr to obtain the title compound.

### Preparation of 1-[1-[1-(4-Methoxy-2-methylphenyl)-6-methyl-2,3-dihydro-1H-pyrrolo[2,3-b]pyridin-4-yl]-1H-pyrazol-3-yl]imidazolidin-2-one

### 1-(1H-pyrazol-3-yl)-2-imidazolidinone

A solution of 3-aminopyrazole (11 kg) in THF (44 L) was treated with 2-chloroethyl isocianate (41.9 Kg). The mixture was aged for 6 hours, then n-Heptane was added over 30 minutes and the mixture cooled to 0-5° C. After 2 hours the suspension was filtered and the cake washed with cold n-Heptane resulting in 34.6 Kg of N-(2-chloroethyl)-3-({[(2-chloroethyl)amino]carbonyl}amino)-1H-pyrazole-1-carboxamide.

The above compound was dissolved in THF and treated with sodium ethoxide 21% wt/wt solution in ethanol over 3 hours. The slurry was aged at R.T. for 24 hours, then cooled to 0-5° C. and aged for 2 additional hours. The suspension was filtered and the cake washed with ethanol (23 L) to obtain 1-(1H-pyrazol-3-yl)-2-imidazolidinone as crude.

The crude was treated with water and aged for 3 hours. The suspension was filtered and the cake washed with water to obtain the title compound (11.96 Kg).

Yield 52% th

### 1-{1-[1-(4-Methoxy-2-methylphenyl)-6-methyl-2,3-dihydro-1H-pyrrolo[2,3-b]pyridin-4-yl]-1H-pyrazol-3-yl}imidazolidin-2-one

To a suspension of CuI (11.36 g) in DMF (2.1 L), trans-N,N′-dimethyl-1,2-diaminocyclohexane (127.29 g) was added under a N₂ atmosphere and the green solution stirred at room temperature for 2-12 h (the colour became green-blue). Then potassium carbonate 325 mesh (1.237 Kg) and 1-(1H-pyrazol-3-yl)-2-imidazolidinone (1.135 Kg) were added followed by the solution of 3-bromo-6-methyl-1-[2-methyl-4-(methyloxy)phenyl]-2,3-dihydro-1H-pyrrolo[2,3-b]pyridine as previously prepared, in DMF (3.0 L). The resulting mixture was heated at 125° C. for 36-42 h. The mixture was then cooled at 60° C. and 4.142 L of water were added dropwise. The suspension was stirred at room temperature for 0.5 h, then the white-brown precipitate was filtered and washed upon the filter with a 1:2 mixture of DMF/H₂O (3.5 L) then with water (3 L). The solid was dried at 80° C. for 24 h.

Yield: 70% th

HPLC greater than 80% a/a

NMR (¹H, CDCl₃): δ 8.29 (d, 1H), 7.15 (d, 1H), 7.04 (s, 1H), 6.85 (d, 1H), 6.79-6.74 (m, 3H), 3.91 (t, 2H), 3.82 (t, 2H), 3.75 (s, 3H), 3.44 (t, 4H), 2.17 (s, 3H), 2.15 (s, 3H)

Structure confirmed by NOE experiment

MS (m/z): 405 [MH]⁺

The crude 1-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone (1.4 Kg) was dissolved at room temperature in a mixture of DCM/MeOH 9:1 (12.6 L). The solution was filtered through a carbon filter washing upon the filter with 4.2 L of the mixture DCM/MeOH 9:1. Then heptane (33.6 L) was added dropwise at room temperature to allow the precipitation of pure DS that was filtered after 2 h of ageing, washed upon the filter with 5.6 L of MeOH and dried at 80° C. for 24 h.

Yield: 67% th

HPLC greater then 98% a/a

### Alternative Crystallisation

The crude 1-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone (933 g) was dissolved at reflux (70-80° C.) in 11.2 L of a mixture anisole/MeOH 7/3. The solution was distilled down to 9.33 L (80° C. jacket, 500 mbar), was brought to room temperature and heptane (18.66 L) was added dropwise to allow the precipitation of the title compound. The pure DS was filtered after 2 h of ageing, washed upon the filter with 3.7 L of heptane and dried at 80° C. for 24 h.

Yield 95% th

HPLC greater then 98% a/a

### Alternative preparation of 1-{1-[1-(4-Methoxy-2-methylphenyl)-6-methyl-2,3-dihydro-1H-pyrrolo[2,3-b]pyridin-4-yl]-1H-pyrazol-3-yl}imidazolidin-2-one starting from 3-iodo-6-methyl-1-[2-methyl-4-(methyloxy)phenyl]-2,3-dihydro-1H-pyrrolo[2,3-b]pyridine

### 1-{1-[1-(4-Methoxy-2-methylphenyl)-6-methyl-2,3-dihydro-1H-pyrrolo[2,3-b]pyridin-4-yl]-1H-pyrazol-3-yl}imidazolidin-2-one

To a suspension of CuI (4.74 g) in DMF (1.0 L), trans-N,N′-dimethyl-1,2-diaminocyclohexane (53.0 g) was added under a N2 atmosphere and the green solution stirred at room temperature for 2-12 h (the colour became green-blue). Then potassium carbonate 325 mesh (515.0 g) and 1-(1H-pyrazol-3-yl)-2-imidazolidinone (472.5 g) were added followed by the solution of 3-iodo-6-methyl-1-[2-methyl-4-(methyloxy)phenyl]-2,3-dihydro-1H-pyrrolo[2,3-b]pyridine as previously prepared, in DMF (1.5 L). The resulting mixture was heated at 90° C. for 15-25 h. The mixture was then cooled at 5oC and 5.0 L of water were added dropwise. The suspension was stirred at 5° C.1-2 h, then the white-brown precipitate was filtered and washed upon the filter with a 1:2 mixture of DMF/H₂O (1.5 L) then with water (1.5 L). The solid was dried at 80oC for 24 h.

Yield: 86% th

HPLC greater than 80% a/a

NMR (1H, CDCl3): 8.29 (d, 1H), 7.15 (d, 1H), 7.04 (s, 1H), 6.85 (d, 1H), 6.79-6.74 (m, 3H), 3.91 (t, 2H), 3.82 (t, 2H), 3.75 (s, 3H), 3.44 (t, 4H), 2.17 (s, 3H), 2.15 (s, 3H)

Structure confirmed by NOE experiment

MS (m/z): 405 [MH]+

The crude 1-(1-6-methyl-1-[2-methyl-4-(methyloxy)phenyl]-2,3-dihydro-1H-pyrrolo[2,3-b]pyridin-4-yl-1H-pyrazol-3-yl)-2-imidazolidinone (447.0 g) was dissolved at room temperature in a mixture of DCM/MeOH 9:1 (5.36 L). The solution was filtered through a carbon filter washing upon the filter with 3.0 L of the mixture DCM/MeOH 9:1. The solution was concentrated to 3.35 L and anisole (6.7 L) was added. The mixture was distilled again to 7.15 L and diluted with methanol (2.86 L). The resulting suspension was finally distilled down to 7.15 L allowing precipitation of pure DS that was filtered after 2-3 h of ageing, washed upon the filter with 1.8 L of anisole and then twice with MeOH (1.8 L). The DS was dried at 80oC for 24 h.

Yield: 73% th

HPLC greater then 98% a/a

### Preparation of 1-[1-(6-Methyl-1-[2-methyl-4-[(trifluoromethyl)oxy]-phenyl]-2,3-dihydro-1H-pyrrolo[2,3-b]pyridin-4-yl)-1H-pyrazol-3-yl]-2-imidazolidin-2-one

CuI (0.02 eq, 3.34 g) was added to a solution of trans-N,N′-dimethyl-1,2-diaminocyclohexane (0.3 eq, 37.4 g) in NMP (0.8 L) under a N₂ atmosphere and the green solution stirred at room temperature for 13 h (the colour became deep-blue). Then potassium carbonate 325 mesh (3.0 eq, 0.363 kg), 1-(1H-pyrazol-3-yl)-2-imidazolidin-2-one (2.5 eq, 0.333 Kg) were added, followed by the solution of 3-Iodo-6-methyl-1-[2-methyl-4-(trifluoromethyloxy)phenyl]-2,3-dihydro-1H-pyrrolo[2,3-b]pyridine in NMP (0.8 L) washing with 0.2 L of NMP. The resulting mixture was heated at 90° C. for 28 h. The mixture was then cooled to 35° C. and CH₂Cl₂ (5.2 L), IPA (1.6 L) and H₂O (3.6 L) were added. The two phases were separated and the organic one washed three times with water (1.6 L)

The organic phase was filter on CUNO filter, washed twice with DCM/IPA 13/4 (1.6 L) then the solution was concentrated to 5.2 L, IPA (4 L) was added and the solution concentrated to 3.2 L. The solution was cooled to 50° C. and seeded. The mixture was cooled to 25° C., stirred at this temperature for 3 hrs. The suspension was filtered and washed with IPA (2×0.8 L). The solid was dried in a vacuum oven at 40° C. for 5-6 hrs

Yield=52%

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A process for preparing compounds of formula (IA) starting from compounds of formula (I) by a coupling reaction catalysed by copper between compounds of formula (I) and a reactive derivative of the upper residue—W-Z 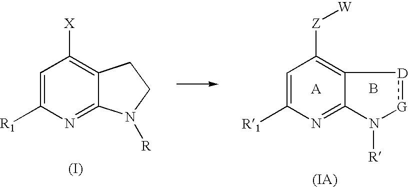
wherein:
R is aryl or heteroaryl, each of which may be substituted by 1 to 4 groups J selected from:
halogen, C1-C6 alkyl, C1-C6 alkoxy, halo C1-C6 alkyl, C2-C6 alkenyl, C2-C6 alkynyl, halo C1-C6 alkoxy, —C(O)R₂, nitro, hydroxy, —NR₃R₄, cyano, and a group Z;
R₁ is hydrogen, C3-C7 cycloalkyl, C1-C6 alkyl, C1-C6 alkoxy, C1-C6 thioalkyl, C2-C6 alkenyl, C2-C6 alkynyl, halo C1-C6 alkyl, halo C1-C6 alkoxy, halogen, NR₃R₄ or cyano;
R₂ is C1-C4 alkyl, —OR₃ or —NR₃R₄;
R₃ is hydrogen or C1-C6 alkyl;
R₄ is hydrogen or C1-C6 alkyl;
R₅ is C1-C6 alkyl, halo C1-C6 alkyl, C1-C6 alkoxy, halo C1-C6 alkoxy, C3-C7 cycloalkyl, hydroxy, halogen, nitro, cyano, —NR₃R₄ or —C(O)R₂;
X is halogen;
R′ corresponds to R;
R′₁ corresponds to R1;
R′₂ corresponds to R2;
R′₃ corresponds to R3;
R′₄ corresponds to R4;
R′₅ corresponds to R5;
R′₆ is C1-C6 alkyl, halo C1-C6 alkyl, C1-C6 alkoxy, halo C1-C6 alkoxy, C3-C7 cycloalkyl, hydroxy, halogen, nitro, cyano, —NR′₃R′₄ or —C(O)R′₂;
R′₇ is hydrogen, C1-C6 alkyl, halogen or halo C1-C6 alkyl;
R′₈ is hydrogen, C3-C7 cycloalkyl, C1-C6 alkyl, C2-C6 alkenyl, C2-C6 alkynyl, NR′₃R′₄ or cyano;
R′₉ is hydrogen, C3-C7 cycloalkyl, C1-C6 alkyl, C2-C6 alkenyl, C2-C6 alkynyl, NR′₃R′₄ or cyano;
R′₁₀ is hydrogen, C3-C7 cycloalkyl, C1-C6 alkyl, C2-C6 alkenyl, C2-C6 alkynyl, NR′₃R′₄ or cyano;
R′₁₁ is hydrogen, C3-C7 cycloalkyl, C1-C6 alkyl, C2-C6 alkenyl, C2-C6 alkynyl, NR′₃R′₄ or cyano;
R′₁₂ is R′₃ or —C(O)R′₂;
D is CR′₈R′₉ or is CR′₈ when double bonded with G;
G is CR′₁₀R′₁₁ or is CR′₁₀ when double bonded with D or is CR′₁₀ when double bonded with X when X is carbon;
W is a 4-8 carbocyclic membered ring, which may be saturated or may contain one to three double bonds, and
in which:
one carbon atom is replaced by a carbonyl or S(O)ₘ; and
one to four carbon atoms may optionally be replaced by oxygen, nitrogen or NR′₁₂, S(O)ₘ, carbonyl, and such ring may be further substituted by 1 to 8 R′₆ groups;
Z is a 5-6 membered heterocycle, which may be substituted by 1 to 8 R′₅ groups; and
m is an integer from 0 to 2.

2. A process, according to claim 1, for preparing the following compounds:
1-{1-[1-(4-Methoxy-2-methylphenyl)-6-methyl-2,3-dihydro-1H-pyrrolo[2,3-b]pyridin-4-yl]-1H-pyrazol-3-yl}imidazolidin-2-one;
1-{1-[1-(4-Methoxy-2-methylphenyl)-6-methyl-2,3-dihydro-1H-pyrrolo[2,3-b]pyridin-4-yl]-1H-pyrazol-3-yl}-3-methylimidazolidin-2-one;
1-{1-[1-(2,4-Dichlorophenyl)-6-methyl-2,3-dihydro-1H-pyrrolo[2,3-b]pyridin-4-yl]-1H-pyrazol-3-yl}imidazolidin-2-one;
1-Acetyl-3-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-Acetyl-3-(1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-(1-{1-[4-(Ethyloxy)-2-methylphenyl]-6-methyl-2,3-dihydro-1H-pyrrolo[2,3-b]pyridin-4-yl}-1H-pyrazol-3-yl)-2-imidazolidinone;
1-[1-(6-Methyl-1-{2-methyl-4-[(1-methylethyl)oxy]phenyl}-2,3-dihydro-1H-pyrrolo[2,3-b]pyridin-4-yl)-1H-pyrazol-3-yl]-2-imidazolidinone;
1-[1-(6-Methyl-1-{2-methyl-4-[(trifluoromethyl)oxy]phenyl}-2,3-dihydro-1H-pyrrolo[2,3-b]pyridin-4-yl)-1H-pyrazol-3-yl]-2-imidazolidinone;
1-(6-{6-Methyl-1-[2-methyl-4-(methyloxy)phenyl]-2,3-dihydro-1H-pyrrolo[2,3-b]pyridin-4-yl}-2-pyridinyl)-2-imidazolidinone;
1-(4-{6-Methyl-1-[2-methyl-4-(methyloxy)phenyl]-2,3-dihydro-1H-pyrrolo[2,3-b]pyridin-4-yl}-2-pyrimidinyl)-2-imidazolidinone;
1-(2-{6-Methyl-1-[2-methyl-4-(methyloxy)phenyl]-2,3-dihydro-1H-pyrrolo[2,3-b]pyridin-4-yl}-4-pyrimidinyl)-2-imidazolidinone;
1-(1-{6-Methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-(3-{6-Methyl-1-[2-methyl-4-(methyloxy)phenyl]-2,3-dihydro-1H-pyrrolo[2,3-b]pyridin-4-yl}phenyl)-2-imidazolidinone;
1-(5-Methyl-1-{6-methyl-1-[2-methyl-4-(methyloxy)phenyl]-2,3-dihydro-1H-pyrrolo[2,3-b]pyridin-4-yl}-1H-pyrazol-3-yl)-2-imidazolidinone;
1-{1-[1-(4-Methoxy-2-methylphenyl)-6-methyl-2,3-dihydro-1H-pyrrolo[2,3-b]pyridin-4-yl]-1H-pyrazol-3-yl}pyrrolidin-2-one;
1-{1-[1-(4-Methoxy-2-methylphenyl)-6-methyl-2,3-dihydro-1H-pyrrolo[2,3-b]pyridin-4-yl]-1H-pyrazol-3-yl}tetrahydropyrimidin-2(1H)-one;
3-(1-{6-Methyl-1-[2-methyl-4-(methyloxy)phenyl]-2,3-dihydro-1H-pyrrolo[2,3-b]pyridin-4-yl}-1H-pyrazol-3-yl)-1,3-oxazolidin-2-one;
Methyl 5-(1-{6-methyl-1-[2-methyl-4-(methyloxy)phenyl]-2,3-dihydro-1H-pyrrolo[2,3-b]pyridin-4-yl}-1H-pyrazol-3-yl)-1,2,5-thiadiazolidine-2-carboxylate 1,1-dioxide);
4-[3-(1,1-Dioxido-1,2,5-thiadiazolidin-2-yl)-1H-pyrazol-1-yl]-6-methyl-1-[2-methyl-4-(methyloxy)phenyl]-2,3-dihydro-1H-pyrrolo[2,3-b]pyridine;
4-[3-(1,1-Dioxido-2-isothiazolidinyl)-1H-pyrazol-1-yl]-6-methyl-1-[2-methyl-4-(methyloxy)phenyl]-2,3-dihydro-1H-pyrrolo[2,3-b]pyridine;
3-Methyl-1-(1-{6-methyl-1-[2-methyl-4-(methyloxy)phenyl]-2,3-dihydro-1H-pyrrolo[2,3-b]pyridin-4-yl}-1H-pyrazol-3-yl)-2(1H)-pyridinone;
2-(1-{6-Methyl-1-[2-methyl-4-(methyloxy)phenyl]-2,3-dihydro-1H-pyrrolo[2,3-b]pyridin-4-yl}-1H-pyrazol-3-yl)-3(2H)-pyridazinone;
1-(1-{6-Methyl-1-[2-methyl-4-(methyloxy)phenyl]-2,3-dihydro-1H-pyrrolo[2,3-b]pyridin-4-yl}-1H-pyrazol-3-yl)-1,3-dihydro-2H-imidazol-2-one;
1-(1-{6-Methyl-1-[2-methyl-4-(methyloxy)phenyl]-1H-pyrrolo[2,3-b]pyridin-4-yl}-1H-pyrazol-3-yl)-2-imidazolidinone;
3-Methyl-4-[6-methyl-4-(3-thiazol-2-yl-pyrazol-1-yl)-2,3-dihydro-1H-pyrrolo[2,3-b]pyridin-1-yl]-benzonitrile; and
1-(4-Methoxy-2-methyl-phenyl)-6-methyl-4-(3-thiazol-2-yl-pyrazol-1-yl)-2,3-dihydro-1H-pyrrolo[2,3-b]pyridine.

3. A process for preparing compounds of formula (I) according to the following Scheme 1: 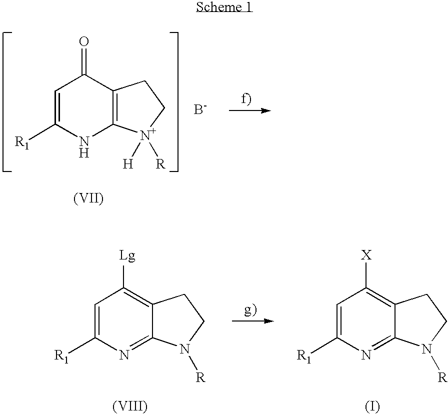
wherein R, R₁, X are defined as in claim 1, and Lg is a leaving group selected among the reactive derivatives of an alkylsulphonic acid;
step f stands for the formation of a reactive derivative of the hydroxy pyridine of compounds (VII); and
step g stands for nucleophilic displacement of the reactive derivative of compounds (VIII) to give the halogenated compounds (I).

4. An intermediate compound of formula (VII) 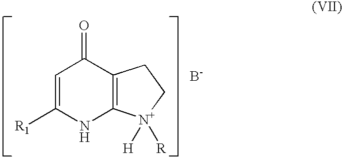
wherein:
R is aryl or heteroaryl, each of which may be substituted by 1 to 4 groups J selected from: halogen, C1-C6 alkyl, C1-C6 alkoxy, halo C1-C6 alkyl, C2-C6 alkenyl, C2-C6 alkynyl, halo C1-C6 alkoxy, —C(O)R₂, nitro, hydroxy, —NR₃R₄, cyano, and a group Z;
R₁ is hydrogen, C3-C7 cycloalkyl, C1-C6 alkyl, C1-C6 alkoxy, C1-C6 thioalkyl, C2-C6 alkenyl, C2-C6 alkynyl, halo C1-C6 alkyl, halo C1-C6 alkoxy, halogen NR₃R₄ or cyano;
R₂ is C1-C4 alkyl, —OR₃ or —NR₃R₄;
R₃ is hydrogen or C1-C6 alkyl;
R₄ is hydrogen or C1-C6 alkyl;
R₅ is C1-C6 alkyl, halo C1-C6 alkyl C1-C6 alkoxy, halo C1-C6 alkoxy, C3-C7 cycloalkyl, hydroxy, halogen, nitro, cyano, —NR₃R₄ or —C(O)R₂; and
Z is a 5-6 membered heterocycle, which may be substituted by 1 to 8 R′₅ groups.

5. A process for the preparation of compounds (IV) starting from compounds of formula (II) and comprising the following steps according to Scheme 2: 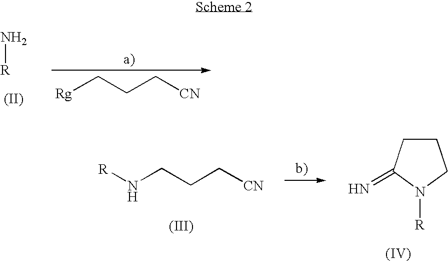
wherein R is defined as in claim 1, Rg is a reactive group selected from: halogen or a reactive derivative of an alkylsulphonic acid;
step a stands for alkylation of the suitable aryl or heteroayl amine of formula (II) with a reactive derivative of butyrronitrile in presence of a base by heating; and
step b stands for the formation of the pyrrolidinone moiety of compounds (IV) which will form the cycle B present in the final compounds (I), by cyclisation of compounds (III), acid catalised and by heating to give the desired compounds (IV).

6. A process for preparing compounds of formula (IVB) according to claim 3 in which step a and step b are performed continuously without isolating intermediate (III), according to the following Scheme 3:

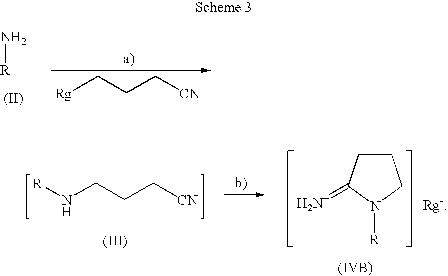

7. An intermediate compound of formula (IVB) 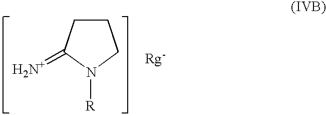
wherein:
R is aryl or heteroaryl, each of which may be substituted by 1 to 4 groups J selected from: halogen C1-C6 alkyl, C1-C6 alkoxy, halo C1-C6 alkyl, C2-C6 alkenyl, C2-C6 alkynyl, halo C1-C6 alkoxy, —C(O)R₂, nitro, hydroxy, —NR₃R₄, cyano, and a group Z;
R₂ is C1-C4 alkyl, —OR₃ or —NR₃R₄;
R₃ is hydrogen or C1-C6 alkyl;
R₄ is hydrogen or C1-C6 alkyl;
R′₅ is C1-C6 alkyl, halo C1-C6 alkyl, C1-C6 alkoxy, halo C1-C6 alkoxy, C3-C7 cycloalkyl, hydroxy, halogen, nitro, cyano, —NR₃R₄ or —C(O)R₂, and
Z is a 5-6 membered heterocycle which may be substituted by 1 to 8 R′₅ groups; and
Rg is a reactive group selected from: halogen or a reactive derivative of an alkylsulphonic acid.

8. A process for the preparation of compounds (VII) starting from compounds of formula (IV) and comprising the following steps: 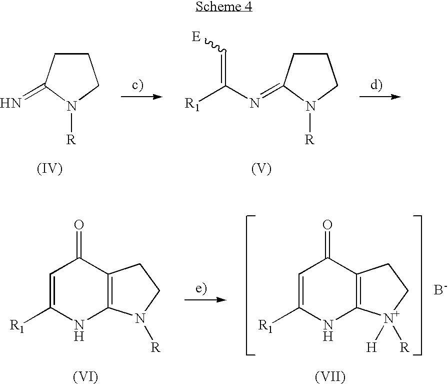
wherein R and R₁, are defined as in claim 1, and
step c stands for a Michael addition of compounds (IV) to a butynoate derivative by heating;
step d stands for cyclisation in basic conditions to give the aromatic compounds (VI); and
step e stands for salt formation by addition of the suitable acid to the compounds (VI).

9. A process for preparing compounds of formula (VII), according to claim 7, starting from compounds of formula (IV) in which compounds (IV) are replaced by compounds (IVB) according to the following Scheme 5: 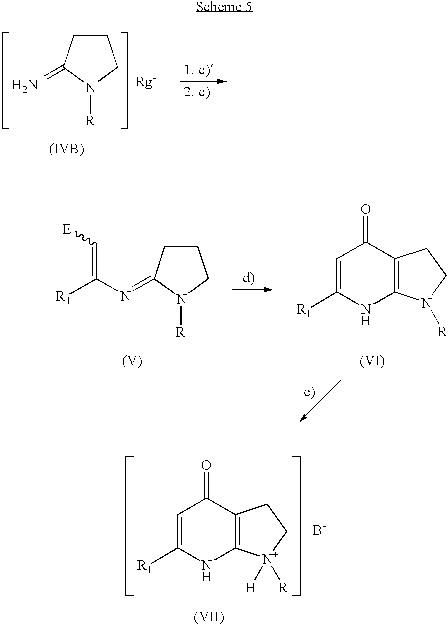
wherein step c′ stands for a basic treatment of compounds (IVB) with a suitable base.
